(19) United States
(12) Patent Application Publication
(10) Pub. No.: US 2018/0042936 A1
(43) Pub. Date: Feb. 15, 2018

(54) MAINTENANCE THERAPY USING TIANEPTINE

(71) Applicant: GENOMIND, Inc., King of Prussia, PA (US)

(72) Inventor: Jay LOMBARD, New York City, NY (US)

(21) Appl. No.: 15/462,101

(22) Filed: Mar. 17, 2017

Related U.S. Application Data
(60) Provisional application No. 62/310,425, filed on Mar. 18, 2016.

(51) Int. Cl. A61K 31/554; A61K 31/135

(52) U.S. Cl. CPC A61K 31/554; A61K 31/135; A61K 2300/00

(57) ABSTRACT

The present application provides a method for treating treatment-resistant depression in a patient in need thereof, comprising administering to said patient a therapeutically effective amount of tianeptine, subsequent to a ketamine treatment; also provided is a method of treating suicidal ideation (SI), post-traumatic stress disorder (PTSD), mild cognitive impairment (MCI) or pre-dementia co-morbid with symptoms of depression; and further provided is maintenance therapy for depression remission.

### BACKGROUND

Treatment-resistant depression occurs in patients suffering from depression who are resistant to known standard pharmacological treatments. A significant percentage of patients with major depression fail treatment with two or more medications.

According to the first phase of the STAR*D study, remission rates were only about 28%, a similar remission rate to that achieved in standard randomized placebo-controlled acute efficacy trials. The STAR*D study is the largest effectiveness study of its kind in “real world” patients, which measured the efficacy of citalopram, a selective serotonin re-uptake inhibitor (“SSRI”) in outpatients with depression (n=2,876). The difficulty of sustaining remission is increasingly apparent for known standard treatments of treatment-resistant depression. The consequences of treatment-resistant depression are profound.

Ketamine was a known medication mainly used for starting and maintaining anesthesia. Common side effects of ketamine include psychological reactions as the medication wears off, including agitation, confusion, or psychosis. Ketamine has been tested in the treatment of breakthrough pain in chronic pain patients (see Carr et al., “Safety and efficacy of intranasal ketamine for the treatment of breakthrough pain in patients with chronic pain: a randomized, double-blind, placebo-controlled, crossover study,” Pain 108 (1-2): 17-27 (2004)).

In more recent years, ketamine has been tested in the treatment of depression. For example, ketamine has been tested in treatment-resistant bipolar disorder, major depressive disorder, and people in a suicidal crisis in emergency rooms (see Tondo et al., “Options for pharmacological treatment of refractory bipolar depression,” Current Psychiatry Reports 16 (2): 431 (2014)).

However, the benefit of ketamine is often of a short duration (see Caddy et al., “Ketamine as the prototype glutamatergic antidepressant: pharmacodynamic actions, and a systematic review and meta-analysis of efficacy,” Therapeutic Advances in Psychopharmacology 4 (2): 75-99 (2014)). Thus, ketamine use over a sustained period of time may be necessary to prevent relapses of depression in many patients. Chronic use of ketamine has been reported to be associated with neurotoxicity and/or toxicities to other organs.

Therefore, there is a need for ketamine responders to have a replacement and/or a maintenance therapy that avoids long term use of ketamine.

### BRIEF SUMMARY

The present disclosure provides a method for treating treatment-resistant depression in a patient in need thereof, comprising administering to the patient a therapeutically effective amount of tianeptine, subsequent to ketamine treatment, wherein the tianeptine treatment is initiated up to about 12 months after the ketamine treatment. The present disclosure also provides a method for treating treatment-resistant depression in a patient in need thereof, comprising administering to the patient a therapeutically effective amount of ketamine, followed by tianeptine treatment, wherein the tianeptine treatment is initiated up to about 12 months after the ketamine treatment. In some embodiments, the depression comprises major depressive disorder or bipolar disorder. In some embodiments, the patient is a responder to the ketamine treatment as measured by a biomarker. In some embodiments, the biomarker is brain-derived neurotrophic factor (“BDNF”).

In some embodiments, the ketamine treatment comprises administering to the patient a dose of between about 0.1 mg/kg body weight/day to about 3.0 mg/kg body weight/day. In some embodiments, tianeptine is administered in an amount of between about 0.1 mg/kg body weight/day to about 10.0 mg/kg body weight/day.

In some embodiments, ketamine or tianeptine is administered in a pharmaceutical composition comprising a therapeutically effective amount of ketamine or tianeptine, and a pharmaceutically acceptable excipient. In some embodiments, the pharmaceutical composition comprises a controlled release composition.

In some embodiments, the tianeptine composition comprises tianeptine sodium, tianeptine hydrochloride, tianeptine phosphate, tianeptine sulfate, tianeptine hemisulfate, tianeptine hemisulfate monohydrate or its crystalline forms in an amount ranging from about 10.0 mg to about 50.0 mg, from about 12.5 mg to about 50.0 mg, from about 25.0 mg to about 50.0 mg, or from about 37.5 mg to about 50.0 mg. In some embodiments, the tianeptine is administered in a daily dose from about 25.0 mg to about 200.0 mg, from about 25.0 mg to about 150.0 mg, from about 25.0 mg to 100.0 mg, or from about 25.0 mg to about 75.0 mg.

The present disclosure also provides a method of treating suicidal ideation (SI), comprising: (1) administering to a subject in need thereof a therapeutically effective amount of ketamine to alleviate symptoms of said suicidal ideation as an emergency treatment; and (2) administering to said subject a therapeutically effective amount of tianeptine as a maintenance treatment. In some embodiments, ketamine is administered in an amount between about 0.1 mg/kg body weight/day to about 3.0 mg/kg body weight/day. In some embodiments, ketamine is administered intranasally.

In some embodiments, tianeptine is administered in an amount of between about 0.1 mg/kg body weight/day to about 10.0 mg/kg bodyweight/day. In some embodiments, tianeptine is administered in a pharmaceutical composition comprising a therapeutically effective amount of tianeptine, and a pharmaceutically acceptable excipient. In some embodiments, the tianeptine pharmaceutical composition comprises a controlled release composition.

In some embodiments, the tianeptine composition comprises tianeptine sodium, tianeptine hydrochloride, tianeptine phosphate, tianeptine sulfate, tianeptine hemisulfate, tianeptine hemisulfate monohydrate or its crystalline forms in an amount ranging from about 10.0 mg to about 50.0 mg, from about 12.5 mg to about 50.0 mg, from about 25.0 mg to about 50.0 mg, or from about 37.5 mg to about 50.0 mg. In some embodiments, the tianeptine is administered in a daily dose from about 25.0 mg to about 200.0 mg, from about 25.0 mg to about 150.0 mg, from about 25.0 mg to 100.0 mg, or from about 25.0 mg to about 75.0 mg.

The present disclosure also provides a method for treating post-traumatic stress disorder (PTSD) in a patient in need thereof, comprising administering to the patient a therapeutically effective amount of tianeptine, subsequent to ketamine treatment, wherein the tianeptine treatment is initiated up to about 12 months after the ketamine treatment. The present disclosure also provides a method for treating post-traumatic stress disorder (PTSD) in a patient in need thereof, comprising administering to the patient a therapeutically effective amount of ketamine, followed by tianeptine treatment, wherein the tianeptine treatment is initiated up to about 12 months after the ketamine treatment.

The present disclosure also provides a method for treating mild cognitive impairment (MCI) or pre-dementia co-morbid with symptoms of depression in a patient in need thereof, comprising administering to the patient a therapeutically effective amount of tianeptine, subsequent to ketamine treatment, wherein the tianeptine treatment is initiated up to about 12 months after the ketamine treatment. The present disclosure also provides a method for treating mild cognitive impairment (MCI) or pre-dementia co-morbid with symptoms of depression in a patient in need thereof, comprising administering to the patient a therapeutically effective amount of ketamine, followed by tianeptine treatment, wherein the tianeptine treatment is initiated up to about 12 months after the ketamine treatment.

The present disclosure further provides a maintenance therapy for depression remission comprising: (a) administering to a patient who suffers from depression a therapeutically effective amount of ketamine for a first predetermined period of time of up to about 12 months; and (b) subsequent to the first predetermined period, administering to the patient in need of maintenance therapy, a therapeutically effective amount of tianeptine.

In some embodiments, ketamine treatment comprises administering to the patient a dose of between about 0.1 mg/kg body weight/day to about 3.0 mg/kg body weight/day. In some embodiments, tianeptine is administered in an amount of between about 0.1 mg/kg body weight/day to about 10.0 mg/kg body weight/day.

In some embodiments, ketamine or tianeptine is administered in a pharmaceutical composition comprising a therapeutically effective amount of ketamine or tianeptine, and a pharmaceutically acceptable excipient. In some embodiments, the pharmaceutical composition comprises a controlled release composition.

In some embodiments, the tianeptine composition comprises tianeptine sodium, tianeptine hydrochloride, tianeptine phosphate, tianeptine sulfate, tianeptine hemisulfate, tianeptine hemisulfate monohydrate or its crystalline forms in an amount ranging from about 10.0 mg to about 50.0 mg, from about 12.5 mg to about 50.0 mg, from about 25.0 mg to about 50.0 mg, or from about 37.5 mg to about 50.0 mg. In some embodiments, the tianeptine is administered in a daily dose from about 25.0 mg to about 200.0 mg, from about 25.0 mg to about 150.0 mg, from about 25.0 mg to 100.0 mg, or from about 25.0 mg to about 75.0 mg.

The present disclosure further provides a kit comprising: (a) a dosage ranging from about 0.1 mg/kg body weight/day to about 3.0 mg/kg body weight/day of ketamine; (b) a dosage ranging from about 0.1 mg/kg body weight/day to about 10.0 mg/kg body weight/day of tianeptine; and (c) instructions for using ketamine and tianeptine for the methods disclosed herein. In some embodiments, the kit further comprising a device comprising a nasal spray inhaler including an aerosol spray ketamine formulation and a pharmaceutically acceptable dispersant.

### DETAILED DESCRIPTION

The headings provided herein are not limitations of the various aspects or aspects of the disclosure, which can be defined by reference to the specification as a whole. Accordingly, the terms defined immediately below are more fully defined by reference to the specification in its entirety. Before describing the present invention in detail, it is to be understood that this invention is not limited to specific methods, compositions or steps, as such can vary.

### I. Definition

In order that the present disclosure can be more readily understood, certain terms are first defined. As used in this application, except as otherwise expressly provided herein, each of the following terms shall have the meaning set forth below. Additional definitions are set forth throughout the application.

The invention includes embodiments in which exactly one member of the group is present in, employed in, or otherwise relevant to a given product or method. The invention includes embodiments in which more than one, or all of the group members are present in, employed in, or otherwise relevant to a given product or method.

Unless defined otherwise, all technical and scientific terms used herein have the same meaning as commonly understood by one of ordinary skill in the art to which this disclosure is related. For example, the definitions of different mental disorders can be found in Diagnostic and Statistical Manual of Mental Disorders, fourth edition (DSM-IV).

In this specification and the appended claims, the singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Comprising or comprise: The terms “comprising” or “comprise” as used herein are synonymous with the terms “include,” “including,” “contain,” and “containing,” and are inclusive or open-ended and do not exclude additional, unrecited elements or method steps.

About: The term “about” as used in connection with a numerical value throughout the specification and the claims denotes an interval of accuracy, familiar and acceptable to a person skilled in the art. Such interval of accuracy is ±10%.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

Treating, treatment, therapy: As used herein, the term “treating” or “treatment” or “therapy” refers to partially or completely alleviating, ameliorating, improving, relieving, delaying onset of, inhibiting progression of, reducing severity of, preventing relapse of, and/or reducing incidence of one or more symptoms or features of a disease, e.g., depression. For example, “treating” depression can refer to alleviating one or more symptoms of depression.

Therapeutically effective amount: As used herein, the term a “therapeutically effective amount” of a drug is an amount effective to demonstrate a desired activity of the drug. For example, a therapeutically effective amount of ketamine is an amount effective for treating as described above.

Dosage amount: The dosage amounts described herein are expressed in amounts of ketamine or tianeptine free base, and do not include the weight of a counterion (e.g., sulfate) or any water or solvent molecules.

A treatment-resistant patient: As used herein, the term “a treatment-resistant patient” means a patient who fails to experience alleviation of one or more symptoms of depression despite undergoing treatment with two or more different antidepressant drugs, and specifically a patient who previously responded to ketamine treatment; or a patient who is unwilling or unable to tolerate the side effects of one or more standard pharmacological or non-pharmacological treatment. A standard treatment may include a serotonin and norepinephrine reuptake inhibitor (“SRNI”), SSRI, an atypical antipsychotic, a dopamine agonist, a mood stabilizer, or other psychotropic as known to those who are skilled in the art.

Treatment-resistant depression: The term “treatment-resistant depression” refers to the depression experienced by a patient who has not previously responded to two or more adequate antidepressant drugs or trials (adequacy of antidepressant trials was determined with the Antidepressant Treatment History Form).

A pharmaceutically acceptable salt: The term a “pharmaceutically acceptable salt” refers to a pharmaceutically acceptable salt by addition of an organic or inorganic acid, or by addition of an organic or inorganic base. The acids include, but are not limited to, hydrochloric hydrobromic, hydroiodic acid, nitric, carbonic, sulfuric, phosphoric, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, 4-hydroxybenzoic, phenylacetic, mandelic, pamoic, methanesulfonic, ethanesulfonic, benzenesulfonic, pantothenic, 2-hydroxyethanesulfonic, toluenesulfonic, sulfanilic, cyclohexylaminosulfonic, stearic, alginic, beta-hydroxybutyric, galactaric or galacturonic acid. The bases include, but are not limited to, NaOH, KOH, LiOH, Ca(OH)₂, Mg(OH)₂, carbonates, bicarbonates triethylamine, benzylamine, diethanolamine, tert-butylamine, dicyclohexylamine, lysine, arginine, N,N′-dibenzylethylenediamine, choline, chloroprocaine, ethylenediamine, meglumine, or procaine.

Tianeptine: As used herein, the term “tianeptine” refers to tianeptine compound, 7-[(3-chloro-6,11-dihydro-6-methyldibenzo[c,f][1,2]thiazepin-11-yl)amino] heptanoic acid S,S-dioxide as shown below:

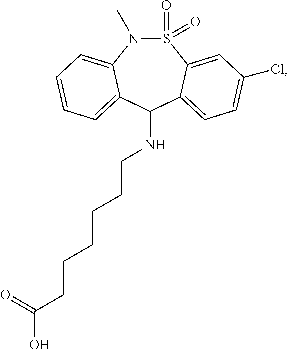 [SMILES: CN1c2ccccc2C(NCCCCCCC(=O)O)c2ccc(Cl)cc2S1(=O)=O]

or a pharmaceutically acceptable salt, or an optical isomer, or a racemic mixture, or a prodrug, or a solvate, or a crystalline form thereof. The pharmaceutically acceptable salts of tianeptine include, for example, tianeptine hemisulfate, tianeptine sulfate, tianeptine hydrochloride, tianeptine phosphate, and tianeptine sodium salt.

Ketamine: As used herein, the term “ketamine” refers to ketamine compound, (RS)-2-(2-Chlorophenyl)-2-(methylamino)cyclohexanone as shown below:

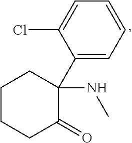 [SMILES: CNC1(c2ccccc2Cl)CCCCC1=O]

or a pharmaceutically acceptable salt, or an optical isomer, or a racemic mixture, or a prodrug, or a solvate, or a crystalline form thereof. The pharmaceutically acceptable salts of ketamine include, for example, ketamine hydrochloride, ketamine sulfate, or ketamine phosphate salts.

### II. Methods

Ketamine is a noncompetitive NMDA receptor antagonist that shows rapid anti-depression efficacy, within 1 hour, in some individuals with treatment-resistant major depressive disorder and bipolar disorder. Brain derived neurotrophic factor (“BDNF”) can serve as a biomarker of treatment response. Studies have demonstrated that ketamine significantly increased plasma BDNF levels in responders as compared to non-responders 240 min post-infusion (see Haile et al., “Plasma brain derived neurotrophic factor (BDNF) and response to ketamine in treatment-resistant depression, Int. J. Neuropsychopharmacol. 17 (2):331-6 (2014)).

Tianeptine is an antidepressant, and its antidepressant effects primarily involve the modulation of glutamatergic neurotransmission and the modulation of the capacity for the brain to exhibit synaptic plasticity.

Without being bound by a particular theory, tianeptine can be used as an anti-depressant for maintenance therapy in patients who have responded to ketamine, as ketamine and tianeptine share common intracellular mechanisms for antidepressant, for example, a specific target for both ketamine and tianeptine related to BDNF.

Unlike ketamine that produces beneficial effects but also produces intolerable dissociative symptoms based upon its effects on NMDA receptors, tianeptine produces an antidepressant effect essentially without dissociative side effects when administered at a therapeutically effective amount.

The present disclosure provides a long term treatment of depression, anxiety and/or other related diseases with tianeptine, subsequent to ketamine treatment. The treatment of present invention maintains remission, and reduces undesirable side effects of ketamine in patients who have responded to ketamine treatment.

In one aspect, the present disclosure provides a method of treating treatment-resistant depression, or a post-traumatic stress disorder (PTSD) in a patient in need thereof, by administering to the patient a therapeutically effective amount of tianeptine, subsequent to ketamine treatment. In some embodiments, the tianeptine treatment is initiated up to about 12 months, about 11 months, about 10 months, about 9 months, about 8 months, about 7 months, about 6 months, about 5 months, about 4 months, 3 about months, about 2 months, about 1 month, about 3 weeks, about 2 weeks, about 1 week, about 6 days, about 5 days, about 4 days, about 3 days, about 2 days, or about 1 day after the ketamine treatment.

In another aspect, the present disclosure provides a method of treating treatment-resistant depression, or a post-traumatic stress disorder (PTSD) in a patient in need thereof, by administering to the patient a therapeutically effective amount of ketamine, followed by tianeptine treatment. In some embodiments, the tianeptine treatment is initiated up to about 12 months, about 11 months, about 10 months, about 9 months, about 8 months, about 7 months, about 6 months, about 5 months, about 4 months, 3 about months, about 2 months, about 1 month, about 3 weeks, about 2 weeks, about 1 week, about 6 days, about 5 days, about 4 days, about 3 days, about 2 days, or about 1 day after the ketamine treatment. In some embodiments, the patient is a responder to the ketamine treatment, as measured by a biomarker BDNF.

Ketamine increases plasma BDNF levels in responders compared to non-responders post ketamine dosing. BDNF can serve as a biomarker to identify whether a patient is a ketamine responder or not. The BDNF level post ketamine dosing can be measured using a BDNF assay as described herein below.

In one aspect, the present disclosure provides a method of treating suicidal ideation (SI), comprising: (1) administering to a subject in need thereof a therapeutically effective amount of ketamine to alleviate symptoms of said suicidal ideation as an emergency treatment; and (2) administering to said subject a therapeutically effective amount of tianeptine as a maintenance treatment. In some embodiments, the patient is a responder to the ketamine treatment, as measured by a biomarker BDNF.

In one aspect, the present disclosure provides a maintenance therapy for depression remission comprising: (a) administering to a patient who suffers from depression a therapeutically effective amount of ketamine for a first predetermined period of time of up to about 12 months, about 11 months, about 10 months, about 9 months, about 8 months, about 7 months, about 6 months, about 5 months, about 4 months, 3 about months, about 2 months, about 1 month, about 3 weeks, about 2 weeks, about 1 week, about 6 days, about 5 days, about 4 days, about 3 days, about 2 days, or about 1 day; and (b) subsequent to the first predetermined period, administering to the patient in need of a maintenance therapy, a therapeutically effective amount of tianeptine. In some embodiments, the patient is a responder to the ketamine treatment, as measured by a biomarker BDNF.

In some embodiments, the patient is a treatment-resistant patient. In some embodiments, the maintenance therapy lasts at least about 1 month, about 2 months, about 3 months, about 4 months, about 5 months, about 6 months, about 1 year, about 2 years, about 3 years, about 4 years, or about 5 years.

Patients with depression commonly experience co-morbid cognitive dysfunction. This dysfunction may be iatrogenic, secondary to medications prescribed for the underlying disorder or due to depression itself. A prolonged use of ketamine may worsen cognitive function. Tianeptine is used as a replacement and/or maintenance therapy for ketamine, which can maintain anti-depressant effects with putative cognitive enhancing effects based upon its action on Ca²⁺/calmodulin-dependent protein kinase type II (“CaMKII”).

Tianeptine may be used as a neuroprotective agent in depressed patients with a high risk of co-morbid cognitive decline. Insufficient neurotrophic support increases the risk for developing Alzheimer's disease (AD), which associates with BDNF and apolipoprotein E (ApoE) with AD. ApoE ε4+/− and ε4+/+ subjects had significantly lower serum BDNF levels than ε4−/− subjects in the whole cohort and the normal control group, suggesting altered BDNF metabolism in ApoE ε4 carriers (see Liu at el., “Associations Between ApoEε4 Carrier Status and Serum BDNF Levels—New Insights into the Molecular Mechanism of ApoEε4 Actions in Alzheimer's Disease,” Mol. Neurobiol. 51 (3): 1271-7 (2015)).

Intra-hippocampal injection of ApoE4, but not ApoE2, attenuated the induction of hippocampal L-LTP in the CA1 region, significantly decreased the levels of phosphorylated CaMKIIα and phosphorylated cAMP response element-binding protein (p-CREB) in the hippocampus, and thus demonstrated that ApoE4 could impair hippocampal L-LTP by reducing p-CaMKIIα and p-CREB.

The effect of ApoE on NMDAR-dependent ERK/CREB signaling is isoform-dependent, and ApoE4 accelerates memory decline in ageing. The up-regulation is accompanied by reduced phosphorylation of AMPA GluR1-S831, CaMKII, and CREB. These findings suggest unique benefits of tianeptine based upon its ability to enhance p-CaMKIIα for ApoE4 in AD risk patients. These patients also include the patients with co-morbid depression and/or BDNF Met allele patients.

In one aspect, the present disclosure provides a method of treating a mild cognitive impairment (MCI), or pre-dementia co-morbid with symptoms of depression in a patient in need thereof, by administering to the patient a therapeutically effective amount of tianeptine, subsequent to ketamine treatment. In some embodiments, the tianeptine treatment is initiated up about 12 months, about 11 months, about 10 months, about 9 months, about 8 months, about 7 months, about 6 months, about 5 months, about 4 months, 3 about months, about 2 months, about 1 month, about 3 weeks, about 2 weeks, about 1 week, about 6 days, about 5 days, about 4 days, about 3 days, about 2 days, or about 1 day after the ketamine treatment. In another aspect, the present disclosure provides a method of treating a mild cognitive impairment (MCI), or pre-dementia co-morbid with symptoms of depression in a patient in need thereof, by administering to the patient a therapeutically effective amount of ketamine, followed by tianeptine treatment. In some embodiments, the tianeptine treatment is initiated about 12 months, about 11 months, about 10 months, about 9 months, about 8 months, about 7 months, about 6 months, about 5 months, about 4 months, 3 about months, about 2 months, about 1 month, about 3 weeks, about 2 weeks, about 1 week, about 6 days, about 5 days, about 4 days, about 3 days, about 2 days, or about 1 day after the ketamine treatment. In some embodiments, tianeptine increases AMPA GluR1 serine-845 phosphorylation. In some embodiments, tianeptine induces CREB phosphorylation. In some embodiments, tianeptine increases BDNF levels.

Depression, anxiety and/or other related diseases to be treated by the present invention include, but are not limited to, major depressive disorder, dysthymic disorder, psychotic depression, postpartum depression, premenstrual syndrome, premenstrual dysphoric disorder, seasonal affective disorder (SAD), anxiety, mood disorder, depressions caused by chronic medical conditions such as cancer or chronic pain, chemotherapy, chronic stress, risk of suicide, and bipolar disorder (or manic depressive disorder), post-traumatic stress disorder (PTSD). It should be understood that depression caused by bipolar disorder may be referred to as bipolar depression. In addition, patients suffering from any form of depression often experience anxiety. The methods of the present invention can be used to treat anxiety or any of the symptoms thereof.

In addition, the methods of the present invention can be used to treat a variety of other neurological conditions. Exemplary conditions include, but are not limited to, a learning disorder, autistic disorder, attention-deficit hyperactivity disorder, Tourette's syndrome, phobia, dementia, AIDS dementia, Alzheimer's disease, Parkinson's disease, Huntington's disease, spasticity, myoclonus, muscle spasm, a substance abuse disorder, urinary incontinence, and schizophrenia.

Also, the method of the present invention can be used to treat depression, wherein the patient does not, and/or has not responded to adequate courses of ketamine, or the patient does not, and/or has not responded to at least two, other antidepressant drugs or therapeutics. In some embodiments, the method of the present invention further comprising (a) identifying a patient as treatment-resistant patient; and (b) administering a therapeutically effective amount of tianeptine to the patient.

In some embodiments, tianeptine of the present invention can be co-administered with one or more other drugs that can ameliorate or exacerbate the symptoms of a neuropsychiatric disorder, including but are not limited to drugs include antidepressants such as lithium salts, carbamazepine, valproic acid, lysergic acid diethylamide (LSD), p-chlorophenylalanine, p-propylidopacetamide dithiocarbamate derivatives e.g., FLA 63; anti-anxiety drugs, e.g., diazepam; monoamine oxidase (MAO) inhibitors, e.g., iproniazid, clorgyline, phenelzine, tranylcypromine, and isocarboxazid; biogenic amine uptake blockers, e.g., tricyclic antidepressants such as desipramine, imipramine and amitriptyline; atypical antidepressants such as mirtazapine, nefazodone, bupropion; serotonin reuptake inhibitors e.g., fluoxetine, venlafaxine, and duloxetine; antipsychotic drugs such as phenothiazine derivatives (e.g., chlorpromazine (thorazine) and triflupromazine)), butyrophenones (e.g., haloperidol (Haldol)), thioxanthene derivatives (e.g., chlorprothixene), S and dibenzodiazepines (e.g., clozapine); benzodiazepines; dopaminergic agonists and antagonists e.g., L-DOPA, cocaine, amphetamine, a-methyl-tyrosine, reserpine, tetrabenazine, benztropine, pargyline; noradrenergic agonists and antagonists e.g., clonidine, phenoxybenzamine, phentolamine, tropolone.

In some embodiments, tianeptine of the present invention can be co-administered with one or more other drugs reported to ameliorate or exacerbate the symptoms of oxidative stress disorder, including but are not limited to drugs include reduced IS glutathione (GSH), glutathione precursors, e.g., N-acetylcysteine; antioxidants, e.g., vitamins E and C, beta carotene and quinones; inhibitors of lipid membrane peroxidation, e.g., 21-aminosteroid U74006F (tirilazad mesylate), and lazaroids; antioxidants such as mazindol; 2c dizocilpine maleate; selegiline; sulfhydryls N-acetyleysteine and cysteamine; dimethylthiourea; EUK-8 a synthetic, low molecular salen-manganese complex; synthetic manganese-based metalloprotein superoxide dismutase mimic, SC52608; free radical scavengers or suppressors, e.g., pegorgotein, tocotrienol, tocopheral, MDL 74,18, LY231617, MCI-186, AVS (nicaraven), allopurinol, rifampicin, oxypurinol, hypochlorous acid or recombinant human Cu, Zn-SOD.

In some embodiments, tianeptine is co-administered with a second therapeutic agent such as those discussed above, simultaneously or sequentially. Tianeptine and the second therapeutic agents are administered in an amount effective to alleviate one or more symptoms of treatment-resistant depression.

The precise time of administration and amount of any particular ketamine and/or tianeptine composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing. The amount of tianeptine in a single dose formulation may vary depending upon the conditions to be treated, and the particular mode of administration.

Treatment may be initiated with smaller dosages which are less than the optimum dose of the compound. Thereafter, the dosage may be increased by small increments until the optimum therapeutic effect is attained.

### Dosage and Regimen

The dosage of any compositions of the present invention may vary depending on the symptoms, age and body weight of the patient, the nature and severity of the disorder to be treated or prevented, the route of administration, and the form of the composition. Any of the compositions may be administered in a single dose or in divided doses. Dosages for the compositions of the present invention may be readily determined by techniques known to those of skill in the art or as taught herein.

In some embodiments, ketamine is formulated with a pharmaceutically acceptable carrier and is administered at a dose of between about 0.01 mg/kg body weight/day to about 3.0 mg/kg body weight/day. In some embodiments, the effective dose of ketamine for depression-alleviation is from about 0.01 to about 1.0 mg/kg of body weight/day, or from about 0.05 to about 0.7 mg/kg of body weight/day, or from about 0.1 to about 0.5 mg/kg of body weight/day.

In some embodiments, ketamine is formulated in an intranasal, transdermal, intravenous, intradermal, or subcutaneous formulations, respectively, which of each may contain total intranasal, transdermal, intravenous, intradermal, or subcutaneous doses of 0.1 mg, 1 mg, 2 mg, 4 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg. Preferably, the effective dose is titrated under the supervision of a physician or medical care provider, so that the optimum dose for the particular application is accurately determined and provided to each individual patient.

In some embodiments, ketamine is administered by intranasal route, and the total dose of ketamine per nasal administration ranges from about 0.1 mg to about 250 mg, from about 1 mg to about 250 mg, from about 10 mg to about 250 mg, from about 25 mg to about 250 mg, from about 50 mg to about 250 mg, from about 75 mg to about 250 mg, from about 100 mg to about 250 mg, from about 125 mg to about 250 mg, from about 150 mg to about 250 mg, from about 200 mg to about 250 mg, about 25 mg, about 50 mg, about 100 mg, about 125 mg, about 150 mg, about 200 mg or about 250 mg.

In some embodiments, ketamine is administered ranging from daily to weekly, daily to monthly, biweekly, or monthly depending on response.

In some embodiments, tianeptine is administered once per day. In other embodiments, tianeptine is administered multiple times per day, for example, two or three times per day. In some embodiments, tianeptine is administered in an amount of from about 0.1 to about 10 mg/kg body weight/day, from about 0.1 to about 5.0 mg/kg body weight/day, or from about 1.0 to about 5.0 mg/kg body weight/day.

In some embodiments, a single tianeptine dose comprises up to about 50.0 mg tianeptine. Preferably, the tianeptine comprises tianeptine sodium salt, tianeptine hemisulfate salt or tianeptine hemisulfate monohydrate. More preferably, the tianeptine comprises tianeptine hemisulfate monohydrate or its crystalline forms.

In some embodiments, daily tianeptine dosages of the invention is in an amount of from about 5.0 mg to about 200.0 mg, from about 10.0 mg to about 200.0 mg, from about 15.0 mg to about 200.0 mg, from about 20.0 mg to about 200.0 mg, from about 25.0 mg to about 200.0 mg, from about 25.0 mg to about 150.0 mg, from about 25.0 mg to 100.0 mg, from about 25.0 mg to about 75.0 mg, from about 25.0 mg to about 50.0 mg, from about 50.0 mg to about 200.0 mg, from about 50.0 mg to about 150.0 mg, from about 50.0 mg to about 100.0 mg, or from about 30.0 mg to about 50.0 mg; about 12.5 mg, 25.0 mg, about 37.5 mg, about 50.0 mg, about 75.0 mg, about 100.0 mg, about 125 mg, about 150 mg, about 175 mg or about 200 mg. Preferably, the tianeptine comprises tianeptine sodium salt, tianeptine hemisulfate salt or tianeptine hemisulfate monohydrate. More preferably, the tianeptine comprises tianeptine hemisulfate monohydrate or its crystalline forms.

In some embodiments, a tianeptine pharmaceutical composition comprises up to about 100.0 mg tianeptine. In some embodiments, an oral tianeptine pharmaceutical composition comprises tianeptine in an amount of from about 0.5 mg to about 50.0 mg, from 5.0 mg to about 50.0 mg, from 7.5 mg to about 50.0 mg, from 10.0 mg to about 50.0 mg, from about 12.5 mg to about 50.0 mg, from about 15.0 mg to about 50.0 mg, from about 17.5 mg to about 50.0 mg, from about 20.0 mg to about 50.0 mg, from about 25.0 mg to about 50.0 mg, from about 30.0 mg to about 50.0 mg, from about 32.5 mg to about 50.0 mg, from about 30.0 mg to about 50.0 mg, from about 37.5 mg to about 50.0 mg, from about 40.0 mg to about 50.0 mg; from 10.0 mg to about 40.0 mg, from about 12.5 mg to about 40.0 mg, from about 15.0 mg to about 40.0 mg, from about 17.5 mg to about 40.0 mg, from about 20.0 mg to about 40.0 mg, from about 25.0 mg to about 40.0 mg, from about 30.0 mg to about 40.0 mg, from about 32.5 mg to about 40.0 mg; from about 12.5 mg to about 37.5 mg, or from about 20.0 mg to about 37.5 mg. Preferably, the tianeptine comprises tianeptine sodium salt, tianeptine hemisulfate salt or tianeptine hemisulfate monohydrate. In some embodiments, an oral pharmaceutical composition comprises tianeptine sodium salt, tianeptine hemisulfate salt or tianeptine hemisulfate monohydrate in an amount of about 5.0 mg, about 10.0 mg, about 12.5 mg, about 15.0 mg, about 20.0 mg, about 25.0 mg, about 30 mg, or about 37.5 mg or about 50 mg.

In some embodiments, the tianeptine pharmaceutical composition of the present invention is a controlled release (or sustained release) composition. In some embodiments, controlled release (or sustained release) composition is a controlled release matrix tablet contains one or more release controlling polymers, such as cellulosic polymers, such as, but are not limited to, hydroxypropyl methylcellulose. More specifically, the one or more release controlling polymers may include a first hydroxypropyl methylcellulose having a viscosity of 80 to 120 cps (2% solution in water) and a second hydroxypropyl methylcellulose having a viscosity of 3,000 to 5,600 cps (2% solution in water). In some embodiments, the first hydroxypropyl methylcellulose and the second hydroxypropyl methylcellulose are present in a ratio of about 2:1 to about 4:1.

In some embodiments, the controlled release matrix tablet further includes a filler, such as, for example, microcrystalline cellulose. The tablet also may further include a lubricant, such as, for example, magnesium stearate. In some embodiments, the tablet also may further include colloidal silica. For a more detailed description of the controlled- or sustained-release systems, see e.g. U.S. Pat. Nos. 5,672,360; 5,968,551; 6,294,195; 7,270,831; and 7,514,100. The controlled release systems can also be prepared by methods known in the art (see e.g., Goodson, in Medical Applications of Controlled Release, vol. 2, pp. 115-138 (1984)). Other controlled release systems discussed in the review by Langer, Science 249:1527-1533 (1990) can be used as well.

In some embodiments, the tianeptine pharmaceutical composition is a controlled release matrix tablet including a pharmaceutically effective amount of tianeptine, particularly tianeptine sodium salt, tianeptine hemisulfate monohydrate or its crystalline forms, and one or more release controlling polymers. The tablet may include about 12.5 mg, about 25 mg, about 37.5 mg, or about 50 mg tianeptine.

In some embodiments, the controlled release matrix tablet has a dissolution rate in vitro, when measured using a USP dissolution apparatus, type II (paddle) at 100 rpm in 900 mL simulated gastric fluid (pH about 1.2) at about 37° C., of less than 14% tianeptine released after 1 hour, between 45% and 80% tianeptine released after 7 hours, and greater than 90% tianeptine released after 16 hours, by weight.

In some other embodiments, the controlled release matrix tablet has a dissolution rate in vitro, when measured using a USP dissolution apparatus, type II (paddle) at 100 rpm in 900 mL simulated gastric fluid (pH about 1.2) at about 37° C., of less than 20% tianeptine released after 2 hours, between 50% and 80% tianeptine released after 8 hours, and greater than 90% tianeptine released after 14 hours, by weight.

In some embodiments, the controlled release matrix tablet, when orally administered to a patient, provides a median time to mean maximum plasma concentration (Tmax) of tianeptine ranging from about 2.0 hours to about 4.0 hours, from about 2.5 hours to about 3.5 hours, or from 2.5 hours to about 3.0 hours. The tablet may include about 12.5 mg, about 25 mg, about 37.5 mg, or about 50 mg tianeptine.

In some embodiments, tianeptine of the present invention is tianeptine sodium salt (see U.S. Pat. Nos. 3,821,249; 5,888,542; 6,441,165; and 6,599,896). In some embodiments, tianeptine of the present invention is a prodrug (see U.S. 2004/0242594 A1). In some embodiments, tianeptine of the present invention is an enantiomer (see U.S. Pat. No. 6,683,072).

In some embodiments, tianeptine of the present invention is tianeptine Form I or Form II (see U.S. Pat. No. 8,367,656). In some embodiments, tianeptine of the present invention is tianeptine phosphate (see U.S. Pat. No. 8,198,268). In some embodiments, tianeptine of the present invention is tianeptine hemisulfate, tianeptine hemisulfate monohydrate, or tianeptine hemisulfate monohydrate crystalline forms (see U.S. Pat. No. 8,198,268).

In other embodiments, tianeptine sodium salt, tianeptine prodrug or tianeptine enantiomer is administered in an immediate release composition or in a controlled release (or sustained release) pharmaceutical composition (see U.S. Pat. Nos. 5,888,542; 6,441,165; 6,599,896; 6,683,072; 8,367,656 and 8,198,268.

In further embodiments, the tianeptine compositions of the present invention can include one or more other therapeutically or pharmacologically active ingredients, such as one or more antidepressants, including but are not limited to, biogenic amine non-selective reuptake inhibitors, e.g., tricyclic antidepressants like Imipramine; serotonin selective reuptake inhibitors like Fluoxetine (Prozac); monoamine oxidase inhibitors (MAO-I) like phenelezine; other types of antidepressant medications including atypical antidepressants.

### Formulations

Ketamine and tianeptine of the present invention may be administered by one or more of a variety of routes, including oral, intravenous, intramuscular, intra-arterial, intramedullary, intrathecal, subcutaneous, intraventricular, trans- or intra-dermal, interdermal, rectal, intravaginal, intraperitoneal, intracardiac, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter.

In some embodiments, a ketamine composition can be administered by a nasal or intranasal route as described in U.S. Pat. Nos. 5,543,434 and 8,785,500. In some embodiments, a tianeptine composition may be administered orally. For a more detailed description of the oral administration of tianeptine, see e.g., U.S. Pat. Nos. 5,888,542; 6,441,165; 6,599,896; 6,683,072; 8,367,656 and 8,198,268.

The present disclosure encompasses the delivery or administration of ketamine or tianeptine compositions described herein by any appropriate route taking into consideration likely advances in the sciences of drug delivery. In general, the most appropriate route of administration will depend upon a variety of factors including the nature of the composition (e.g., its stability in various bodily environments such as the bloodstream and gastrointestinal tract), the condition of the patient (e.g., whether the patient is able to tolerate particular routes of administration), etc. For application by the ophthalmic mucous membrane route, tianeptine composition of the present invention may be formulated as eye drops or eye ointments.

These formulations may be prepared by conventional means, and, if desired, the compositions may be mixed with any conventional additives/excipients, including but are not limited to a binder, a disintegrating agent, a lubricant, a release control agent, a solubilizing agent, a suspension aid, an emulsifying agent, a coating agent, a sweetening agent, a flavoring agent, a perfuming agent, a colorant, a preservative or an antioxidant.

In formulations of the present invention, wetting agents, emulsifiers and lubricants, such as sodium lauryl sulfate and magnesium stearate, as well as coloring agents, release agents, coating agents, sweetening, flavoring and perfuming agents, preservatives and antioxidants may be present in the formulated agents.

Compositions of the present invention may be suitable for oral, topical (including buccal and sublingual), rectal, vaginal, aerosol and/or parenteral administration. The formulations may conveniently be presented in unit dosage form and may be prepared by any methods well known in the art of pharmacy.

Methods of preparing these formulations include the step of bringing into association compositions of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of an active ingredient. Compositions of the present invention may also be administered as a bolus, electuary, or paste.

In solid dosage forms for oral administration (capsules, tablets, pills, dragees, powders, granules and the like), an active agent is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent. Formulations which are suitable for vaginal administration also include pessaries, tampons, creams, gels, pastes, foams or spray formulations containing such carriers as are known in the art to be appropriate.

Dosage forms for transdermal administration of a subject composition includes powders, sprays, ointments, pastes, creams, lotions, gels, solutions, and patches.

For topical ocular administration compositions of this invention may take the form of solutions, gels, ointments, suspensions or solid inserts, formulated so that a unit dosage comprises a therapeutically effective amount of the active component or some multiple thereof in the case of a combination therapy.

Pharmaceutical compositions of this invention suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

### Kits

Also within the scope of the present disclosure are kits that comprise ketamine and tianeptine. Kits typically include a label indicating the intended use of the contents of the kit and instructions for use. The term “label” includes any writing, or recorded material supplied on or with the kit, or which otherwise accompanies the kit. Accordingly, this disclosure provides a kit for treating a patient inflicted with depression, PTSD, SI, mild cognitive impairment (MCI), or pre-dementia co-morbid with symptoms of depression, the kit comprising: (a) a dosage ranging from about 0.1 to about 3.0 mg/kg body weight/day of ketamine; (b) a dosage ranging from about 0.1 to about 10.0 mg/kg body weight/day of tianeptine; and (c) instructions for using ketamine and tianeptine in any of the methods disclosed herein. In some embodiments, ketamine is administered intranasally. In some embodiments, the kit contains up to about 30, about 60 or about 90 daily dosages of tianeptine. In some embodiments, ketamine is administered intranasally. In some embodiments, the kits contain a device for patient self-administration of ketamine comprising a nasal spray inhaler containing an aerosol spray formulation of ketamine and a pharmaceutically acceptable dispersant, wherein the device is metered to disperse an amount of the aerosol formulation by forming a spray that contains a therapeutically effective dose of ketamine.

All U.S. and foreign patents, patent application publications, and other publications cited herein are fully incorporated by reference herein in their entirety.

The present invention is further illustrated by the following examples which should not be construed as further limiting.

### EXAMPLES

### Example 1

### BDNF Assay

A blood sample is obtained at baseline and at post-ketamine administration. Whole blood samples are collected in vacutainer tubes containing EDTA then centrifuged at 3000 r/min for 15 min. Plasma supernatant is then transferred to a new sterile microfuge tube and sample stored at −80 ° C. until processed for BDNF. Samples are processed within 1 h of being collected to decrease variability. BDNF concentrations are quantitatively determined by enzyme-linked immunosorbent assay (ELISA) according to the manufacturer's instructions (DuoSet ELISA Development Kit R&D Systems, Minneapolis, USA). Samples are diluted 1:20 in sample diluent buffer, then aliquotted onto 96 well plates coated with a monoclonal antibody raised against BDNF. BDNF standards (human) and plasma samples are assayed in duplicate. Plates are incubated then washed with buffer (1×PBS). BDNF conjugated to horseradish peroxidase is then added. Following additional washing, substrate solution followed by a stop solution is added to halt the reaction. Absorbance is determined at 450 nm using a Multiskan FC plate reader (Thermo Fischer Scientific Inc., USA), with the correction wavelength set at 540 nm. A standard curve is constructed by plotting the mean absorbance for each standard against BDNF concentration. The data is then linearized by plotting the log of the BDNF concentration vs. the log of the O.D. and the best fit line is determined by regression analysis. BDNF concentration generated in duplicate is averaged to give a value in ng/ml after correcting for sample dilution factor and subtraction of background (blank from standard curve). Ketamine increases plasma BDNF levels in responders compared to non-responders post-infusion of ketamine. BDNF severs as a biomarker to identify whether an individual is a ketamine responder or not.

### Example 2

### Tianeptine Hemisulfate Monohydrate

Tianeptine sodium (100 grams) is dissolved in 50:50 isopropanol:water (500 milliliters) at room temperature to give a colorless solution. The solution is filtered and to it is added 45.4% sulfuric acid in water (73.2 milliliters). Crystalline tianeptine hemisulfate monohydrate is completely crystallized within 2 hours at which point the mixture is filtered. The solid is washed with 50:50 isopropanol:water (500 mL) and water (300 mL), and then allowed to dry under ambient conditions overnight, and the resulting tianeptine hemisulfate monohydrate comprises about 1:0.5:1 ratio of ionized tianeptine:sulfate counterion:water.

### Example 3

### Tianeptine Sodium Tablet Formulation

A tianeptine tablet containing tianeptine sodium can be prepared using the formula given in Table 1.

| Components | Quantities (mg per unit formula) |
|---|---|
| Tianeptine sodium salt | 50 |
| Calcium hydrogen phosphate dihydrate | 50 |
| Lactose monohydrate | 96.4 |
| Methylhydroxypropylcellulose | 100 |
| Anhydrous colloidal silica | 0.6 |
| Magnesium stearate | 3 |
| Coating composition | 15 |

### Example 4

### Tianeptine Hemisulfate Monohydrate Tablet Formulation

A controlled release tianeptine tablet containing tianeptine hemisulfate monohydrate can be prepared using the formula given in Table 2.

| Components | Quantities (mg per unit formula) |
|---|---|
| Tianeptine hemisulfate monohydrate | 28.84 |
| Avicel PH200 | 60.0 |
| Methocel K100 LV CR Premium | 81.5 |
| Methocel K4M CR Premium | 27.16 |
| colloidal silica | 0.5 |
| Magnesium stearate | 2.0 |

1. A method for treating treatment-resistant depression in a patient in need thereof, comprising administering to the patient a therapeutically effective amount of tianeptine, or a pharmaceutically acceptable salt thereof, subsequent to ketamine, or a pharmaceutically acceptable salt thereof, treatment, wherein the tianeptine, or a pharmaceutically acceptable salt thereof, treatment is initiated up to about 12 months after the ketamine, or a pharmaceutically acceptable salt thereof, treatment.

2. A method for treating treatment-resistant depression in a patient in need thereof, comprising administering to the patient a therapeutically effective amount of ketamine, or a pharmaceutically acceptable salt thereof, followed by tianeptine, or a pharmaceutically acceptable salt thereof, treatment, wherein the tianeptine, or a pharmaceutically acceptable salt thereof, treatment is initiated up to about 12 months after the ketamine, or a pharmaceutically acceptable salt thereof, treatment.

3. The method of claim 1, wherein the patient is a responder to the ketamine, or a pharmaceutically acceptable salt thereof, treatment.

4. The method of claim 3, wherein the patient's response to the ketamine, or a pharmaceutically acceptable salt thereof, treatment is measured by detecting a biomarker.

5. The method of claim 4, wherein the biomarker is brain-derived neurotrophic factor (BDNF).

6. The method of claim 1, wherein the ketamine, or a pharmaceutically acceptable salt thereof, treatment comprises administering to the patient a dose of about 0.1 mg/kg/day to about 3.0 mg/kg/day.

7. The method of claim 1, wherein tianeptine, or a pharmaceutically acceptable salt thereof, is administered in an amount of about 0.1 mg/kg/day to about 10.0 mg/kg/day.

8-10. (canceled)

11. The method of claim 1, wherein the tianeptine comprises tianeptine hemisulfate monohydrate or its crystalline forms.

12. The method of of claim 1, wherein the tianeptine, or a pharmaceutically acceptable salt thereof, is administered in a daily dose from about 25.0 mg to about 200.0 mg, from about 25.0 mg to about 150.0 mg, from about 25.0 mg to 100.0 mg, or from about 25.0 mg to about 75.0 mg.

13. (canceled)

14. A method for treating suicidal ideation (SI), comprising: (1) administering to a subject in need thereof a therapeutically effective amount of ketamine, or a pharmaceutically acceptable salt thereof, to alleviate symptoms of said suicidal ideation as an emergency treatment; and (2) administering to said subject a therapeutically effective amount of tianeptine, or a pharmaceutically acceptable salt thereof, as a maintenance treatment.

15. The method of claim 14, wherein ketamine, or a pharmaceutically acceptable salt thereof, is administered in an amount of about 0.1 mg/kg/day to about 3.0 mg/kg/day.

16. The method of claim 14, wherein ketamine, or a pharmaceutically acceptable salt thereof, is administered intranasally.

17. The method of claim 14, wherein tianeptine, or a pharmaceutically acceptable salt thereof, is administered in an amount of about 0.1 mg/kg/day to about 10.0 mg/kg/day.

18-20. (canceled)

21. The method of claim 14, wherein the tianeptine comprises tianeptine hemisulfate monohydrate or its crystalline forms.

22. The method of claim 14, wherein the tianeptine, or a pharmaceutically acceptable salt thereof, is administered in a daily dose from about 25.0 mg to about 200.0 mg, from about 25.0 mg to about 150.0 mg, from about 25.0 mg to 100.0 mg, or from about 25.0 mg to about 75.0 mg.

23-54. (canceled)
